(19) United States
(12) Patent Application Publication
(10) Pub. No.: US 2026/0110656 A1
(43) Pub. Date: Apr. 23, 2026

(54) ELECTROCHEMICAL SENSOR PREPARED FROM NIOBIUM CARBIDE-REDUCED GRAPHENE OXIDE AEROGEL COMPOSITE MATERIAL AND ITS UTILIZATION

(71) Applicant: NATIONAL TAIPEI UNIVERSITY OF TECHNOLOGY, Taipei City (TW)

(72) Inventor: Kuo-Yuan HWA, Taipei City (TW)

(73) Assignee: NATIONAL TAIPEI UNIVERSITY OF TECHNOLOGY, Taipei City (TW)

(21) Appl. No.: 18/927,769

(22) Filed: Oct. 25, 2024

Related U.S. Application Data
(60) Provisional application No. 63/708,959, filed on Oct. 18, 2024.

(51) Int. Cl. G01N 27/30; G01N 33/18

(52) U.S. Cl. CPC G01N 27/308; G01N 33/1826

(57) ABSTRACT

The present invention provides a composite material NC/rGO-A, comprising niobium carbide (NC) and reduced graphene oxide aerogel (rGO-A), designed for modification onto a screen-printed carbon electrode (SPCE). Also provided is an NC/rGO-A-modified SPCE (NC/rGO-A/SPCE) with excellent electrical conductivity. This modified electrode is highly effective for detection of organic pollutants in a contaminated water sample, ensuring high repeatability, reproducibility, and stability.

### CROSS-REFERENCE TO RELATED APPLICATION

This application claims the benefit of U.S. provisional application No. 63/708,959, filed Oct. 18, 2024 under 35 U.S.C. § 119, the entire content of which is incorporated herein by reference.

### FIELD OF THE INVENTION

The present invention relates to an electrochemical sensor, such as an electrode, for detection of organic pollutants in waste water, particularly a new composite material combined with niobium carbide (NC) and reduced graphene oxide aerogel (rGO-A) based modified electrode, and a method for detection of nitroanilines using the same.

### BACKGROUND OF THE INVENTION

The increase in population and industries results in the contamination of ecosystems and aquatic environments by industrial organic wastes (Baran et al., 2023; Chakraborty et al., 2018; Das et al., 2020; Karunanayake et al., 2016). Although the fact that organizations utilize safety management systems when analyzing wastewater, certain amounts of it are disposed of beyond their limits (Fazzo et al., 2017). Likewise, aromatic amines are essential because they act as raw ingredients for numerous industries.

Nitroanilines (NTA) are known organic pollutants that have attracted a great deal of interest due to their harmful behavior and carcinogenic potential. NTA including 2-Nitroanline (2-NTA), 3-Nitroaniline (3-NTA), and 4-Nitroaniline (4-NTA) are soluble pollutants in waste water, which are harmful contaminant and commonly utilized in the azo dyes, synthesis of pesticides, pharmaceuticals, antioxidants, polymers, and anti-corrosive materials (Manavalan et al., 2019; Pfeifer et al., 2016; Tong et al., 2010). Such 4-NTA components quickly penetrate the groundwater, wastewater, and soil. The US Environmental Protection Agency (EPA) has recognized and categorized 4-NTA as an essential pollutant, and this compound has been recorded as a contaminant throughout the environment at levels up to 100 mg L⁻¹ (de Barros et al., 2021; Silambarasan and Vangnai, 2016). Long-term usage of contaminated water can cause mutagenic and carcinogenic effects in people. Considering these factors, several developed and developing countries examined nitroaniline isomers as major pollutants.

There are different analytical techniques employed for the detection and determination of 4-NTA in waste water, including high-performance liquid chromatography, photodegradation, advanced oxidation process, capillary electrophoresis, and spectrophotometry methods (Gautam et al., 2005; Guo et al., 2006; Neyens and Baeyens, 2003; Niazi et al., 2007; Tong et al., 2010). Among them, the electrochemical techniques are commonly used for detection of NTA, such as an electrochemical sensor, because it is simple, of low costs, and precise, with high selectivity, high accuracy and sensitivity. Also, the disadvantages in other techniques like less sensitivity, high manpower, and more time-consuming sample preparation can all be avoided (Nataraj et al., 2022; Palpandi and Raman, 2020; Yamuna et al., 2021b).

In recent years the outstanding progress of two dimensional (2D) materials has been achieved with transition metal carbides (TMCs), nitrides, phosphides, and chalcogenides, which have high electronic conductivity, earth-abundant, good corrosion resistance, and have great stability (Hwu and Chen, 2005; Yuan et al., 2020). TMCs have high catalytic activity, which will help them attain superior electrochemical performances (Weidman et al., 2012). TMCs such as titanium carbide, molybdenum carbide, tungsten carbide, vanadium carbide, and niobium carbide have sparked attention in different electrochemical applications like sensors, energy storage, water splitting and etc., (Gao et al., 2019; Kimmel et al., 2014; Kokulnathan et al., 2023, 2021b, 2020; Kokulnathan and Wang, 2020; Santhan and Hwa, 2023).

Among all the TMCs, niobium carbide (NC) has excellent chemical stability, physical properties, a huge quantum capacitance, outstanding electrical conductivity (2.9×10⁶ S/m), high melting point (3610° C.), and corrosion resistance (Coy et al., 2017; Grove et al., 2010; Qin et al., 2021; Wang et al., 2021). NC is preferable for electrode materials in electrochemical and catalytic activities (Santhan and Hwa, 2022a). However, NC will show low electrical resistance at room temperature which is as small as 4.6 uΩ cm as well as cubic NC will exhibit superconductivity at below 12 K (Klug et al., 2011; Mahle et al., 2022).

Graphene became a widely investigated 2-dimensional material because of its unique properties which are superior thermal conductivity (4840-5300 W m⁻¹ K⁻¹), higher theoretical surface area (2600 m² g⁻¹), high electron mobilities (2×10⁵ cm² V⁻¹ s⁻¹), and high mechanical properties (tensile strength up to 130 GPa, an elastic modulus of 1000 GPa) (Ahmed et al., 2023; Cheng et al., 2017; Guex et al., 2017; Kumar et al., 2018). A honeycomb-shaped structure of carbon atoms (sp² hybridized) having a thickness that is similar to one carbon atom renders up graphene. Graphene oxide (GO) is produced by oxidation of graphite, which provides oxygen-containing functional groups on the graphite surface such as hydroxyl and carboxyl groups. Nevertheless, owing to the existence of these functional groups, GO cannot be active.

The oxygen functional groups in graphene oxide have been eliminated to produce reduced graphene oxide (rGO). There are various techniques for performing the reducing method, including chemical, or electrochemical reduction, and thermal. The outcome of rGO is an extremely conductive compound with outstanding electrochemical characteristics that can be used in a wide range of applications, including sensors, water splitting, photocatalysis and energy storage devices (Caliskan et al., 2022; Cheng et al., 2017; Dong et al., 2021; Halankar et al., 2021; Li et al., 2019; Manna and Raj, 2018; Tong et al., 2018; Zakaria et al., 2020; Zhang et al., 2022; Zhao et al., 2016). Overall, owing to its more effective electrical conductivity, large surface area, and adaptability in functionalization, rGO is considered as an intriguing material for a variety of electrochemical applications (Hwa et al., 2021; Nataraj et al., 2020; Nataraj and Chen, 2021; Santhan et al., 2022; Selvi et al., 2020).

Rather than employing only rGO, it can be utilized as aerogel which contributes both the significances of rGO and aerogel. The material with open interconnected porosity, high specific surface area, and low density is the aerogel. The rGO aerogels (rGO-A) were some advantageous with the above-mentioned properties along with good electrical/thermal conductivity, good mechanical strength and lightweight. The rGO-A enables electron transfer pathways, rich active sites resulting with good electrochemical performances. However, there are still some structural defects existing in rGO-A, the repeatability, reproducibility, and stability of rGO-A based modified electrode are still need to be improved.

Therefore, it is still desirable to develop a new modified electrode for detection of environmental pollutants in waste water.

### SUMMARY OF THE INVENTION

It is unexpectedly discovered in the present invention that a novel composite material combined with niobium carbide (NC) and reduced graphene oxide aerogel (rGO-A), also called as NC/rGO-A, is prepared and can be applied to modify a screen-printed carbon electrode (SPCE) for detection of organic pollutants in contaminated water sample with high repeatability, reproducibility, and stability, and with low interfering.

In one aspect, the present invention provides a composite material, called as NC/rGO-A, comprising niobium carbide (NC) and reduced graphene oxide aerogel (rGO-A) at a ratio of about 1:1.

In one example of the present invention, the niobium carbide (NC) has characteristic diffraction peaks at diffraction angle (20) of 35.1°, 40.6°, 58.5°, 69.9°, and 73.5°.

In one example of the present invention, the reduced graphene oxide aerogel (rGO-A) has characteristic diffraction peaks at diffraction angle (20) of 25.4° and 43.5°.

In one example of the present invention, the composite material NC/rGO-A comprises
50%-60% of niobium,
20%-30% carbon, and
10%-20% of oxygen.

In one particular example of the present invention, the composite material NC/rGO-A comprises
56.9% of niobium,
29.6% carbon, and
13.5% of oxygen.

In one further aspect, the present invention provides a method for preparing the composite material NC/rGO-A, comprising the steps of:
(i) dissolving niobium carbide and reduced graphene oxide aerogel in water as a mixture;
(ii) stirring to homogenize the mixture; and
(iii) sonicating the mixture thoroughly to produce the composite material NC/rGO-A.

In one yet aspect, the present invention provides a NC/rGO-A based modified electrode, called as NC/rGO-A/SPCE that has great electrical conductivity.

In one yet yet aspect, the present invention provides an NC/rGO-A based modified electrode having great electrical conductivity, comprising the steps of:
(i) rinsing a bare screen-printed carbon electrode in distilled water and ethanol to obtain a rinsed bare screen-printed carbon electrode;
(ii) coating the composite material NC/rGO-A onto the rinsed screen-printed carbon electrode obtained in step (i) to obtain a screen-printed carbon electrode with NC/rGO-A coating; and
(iii) drying the screen-printed carbon electrode with NC/rGO-A coating as obtained in step (ii).

In one example of the present invention, the screen-printed carbon electrode is dried in a hot air oven at about 50° C. in step (iii).

In a further yet aspect, the present invention provides an NC/rGO-A based modified electrode, called as NC/rGO-A/SPCE, comprising an electrode coated with the composite material NC/rGO-A according to the invention, which is prepared by the method mentioned above.

According to the present invention, the NC/rGO-A/SPCE is used for detection of organic pollutants in a contaminated water sample.

According to the present invention, the NC/rGO-A/SPCE is of high repeatability, reproducibility, and stability for detection of organic pollutants in a contaminated water sample.

In some embodiment of the invention, the NC/rGO-A based modified electrode has an excellent selectivity exposing to interfering compounds, such as 4-nitrophenol, aminophenol, nitrobenzene, acetaminophen, carbendazim, chlorine ions, sodium ions, glucose, mercury, and hydroquinone.

In some examples of the present invention, the organic pollutants are nitroanilines (NTA), including 2-Nitroaniline (2-NTA), 3-Nitroaniline (3-NTA), and 4-Nitroaniline (4-NTA).

In one particular example of the present invention, the organic pollutant is 4-NTA.

In the present invention, in wherein the contaminated water sample is collected from industrial waste water, river water, or lake water

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIGS. 1A-1B show an abstract of the present invention. FIG. 1A shows the synthesis scheme of NC, rGO-A, NC/rGO-A and electrochemical application; FIG. 1B shows the graphical abstract illustrating the use of NC/rGO-A/SPCE for detecting 4-NTA in environmental real samples by using DPV method.

FIGS. 2A-2B show the XRD analyzing results of three materials, including NC, rGO-A, and NC/rGO-A. FIG. 2A shows the X-ray diffraction spectra of NC- (blue color spectrum), rGO-A- (orange color spectrum), and NC/rGO-A- (pink color spectrum); FIG. 2B shows the XRD spectrum of NC with stick pattern (insert crystal structure of NC).

FIG. 3 shows the Raman spectra of NC- (blue color spectrum), rGO-A- (orange color spectrum), and NC/rGO-A- (pink color spectrum).

FIG. 4 shows the FTIR spectra of NC- (blue color spectrum), rGO-A- (orange color spectrum), and NC/rGO-A- (pink color spectrum).

FIGS. 5A-5D show the XPS analysis result of construction of NC/rGO-A. FIG. 5A shows the XPS survey plot of NC/rGO-A; FIG. 5B shows the XPS spectra for Nb 3d; FIG. 5C shows the XPS spectra for C Is; FIG. 5D shows the XPS spectra for O 1s.

FIG. 6 shows the pictures of materials comparisons in morphology using TEM and FESEM, wherein the materials include NC, rGO-A, and NC/rGO-A.

FIGS. 7A-7C show the FESEM elemental mapping, EDX spectrum and pie chart of NC (FIG. 7A), rGO-A (FIG. 7B) and NC/rGO-A (FIG. 7C).

FIGS. 8A-8F show the results of electrochemical resistance and surface area analysis. FIG. 8A shows electrochemical impedance spectroscopy (EIS) spectrums of bare SPCE, NC/SPCE, rGO-A/SPCE and NC/rGO-A/SPCE in 5 mM [Fe(CN)₆]^{3-/4-} and 0.1 M of KCl electrolyte solution with 1 Hz to 100 kHz; FIG. 8B shows the histogram of EIS analysis; FIG. 8C shows the cyclic voltammetry (CV) graphs of bare SPCE, NC/SPCE, rGO-A/SPCE and NC/rGO-A/SPCE in 5 mM [Fe(CN)₆]^{3-/4-} and 0.1 M of KCl electrolyte solution with scan rate 50 mV/s; FIG. 8D shows the histogram of the CV; FIG. 8E shows the profiles of scan rate analysis (20 m V/s to 200 m V/s) at NC/rGO-A/SPCE in 5 mM [Fe(CN)₆]^{3-/4-} and 0.1 M of KCl electrolyte; FIG. 8F shows the linear plot of scan rate vs current.

FIGS. 9A-9F show the results of electrochemical detection of 4-NTA. FIG. 9A shows the cyclic voltammetry of bare SPCE, NC/SPCE, rGO-A/SPCE and NC/rGO-A/SPCE with 100 μM addition 4-NTA with the presence of 0.1 M PBS at scan rate 50 mV/s; FIG. 9B shows the histogram plots of modified electrode vs current; FIG. 9C shows the CV profiles of NC/rGO-A/SPCE with 100 μM concentration of 4-NTA and 0.1 M PBS with different scan rate from 20 to 200 m V/s; FIG. 9D shows the linear plot of square root of different scan rates vs current; FIG. 9E shows the CV images of NC/rGO-A/SPCE at different pH (3.0 to 11.0) with 100 μM 4-NTA studied in 0.1 M PBS with scan rate 50 m V/s; FIG. 9F shows the linear plot of different pH vs potential.

FIG. 10 shows the scheme of electrochemical reduction mechanism of 4-nitroaniline.

FIGS. 11A-11D show the results of different concentrations analysis. FIG. 11A shows the CV images of NC/rGO-A/SPCE with the different concentration (25 μM to 200 μM) of 4-NTA with 0.1 M PBS and scan rate 50 mV/s; FIG. 11B shows the linear plot of different concentration vs current; FIG. 11C shows the DPV images of NC/rGO-A/SPCE with the (0.039 μM to 1602 μM) concentration of 4-NTA and 0.1 M PBS; FIG. 11D shows the calibration plot of 4-NTA concentration vs current.

FIGS. 12A-12B show the results of interference analysis. FIG. 12A shows the DPV curves of NC/rGO-A/SPCE with the 100 μM 4-NTA solution with different interferents; FIG. 12B shows the relative error bar of selectivity analysis.

FIGS. 13A-13F show the results of cyclic voltammetry curves studied at NC/rGO-A with 100 μM of 4-NTA. FIGS. 13A and 13B show the CV curves and the histogram plot of repeatability; FIGS. 13C and 13D show the CV curves and the histogram plot of reproducibility; FIGS. 13E and 13F show the CV curves and the histogram plot of stability.

FIGS. 14A-F show the result of monitoring the 4-NTA vs current in different water samples. FIGS. 14A and 14B show the DPV images and linear plot of industrial river water; FIGS. 14C and 14D show the DPV images and linear plot of waste water; FIGS. 14E and 14F show the DPV images and linear plot of lake water.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an” and “the” include plural references unless explicitly indicated otherwise. Thus, for example, reference to “a sample” includes a plurality of such samples and their equivalents known to those skilled in the art.

As used herein, the term “about” generally refers to a range slightly above or below the stated value, allowing for minor deviations. The exact scope of “about” depends on the context of the invention and can vary depending on factors such as the precision of the technology involved or industry standards.

As used herein, the term “SPCE” refers to screen-printed carbon electrode (SPCE) which are a widely used type of electrochemical sensor due to their affordability, ease of production, and versatility in various applications, especially in analytical chemistry, biosensing, and environmental monitoring. SPCEs are fabricated through a screen-printing process, where conductive carbon-based inks are deposited onto a substrate, usually made of ceramics or plastics. This printing technique enables the mass production of reproducible and cost-effective electrodes, suitable for both research and commercial purposes. A variety of materials can be applied to SPCE, as used herein, the materials NC, rGO-A, and NC/rGO-A are applied to SPCE and were represented as NC/SPCE, rGO-A/SPCE, and NC/rGO-A/SPCE.

As used herein, the term nitroanilines (NTA), include 2-Nitroaniline (2-NTA), 3-Nitroaniline (3-NTA), and 4-Nitroaniline (4-NTA).

The present invention provides a novel composite material NC/rGO-A, which may be prepared by combining niobium carbide (NC) and reduced graphene oxide aerogel (rGO-A) at a ratio of about 1:1 through a sonication process. The NC/rGO-A can be applied to modify a SPCE for detection of organic pollutants in a contaminated water sample with high repeatability, reproducibility, and stability.

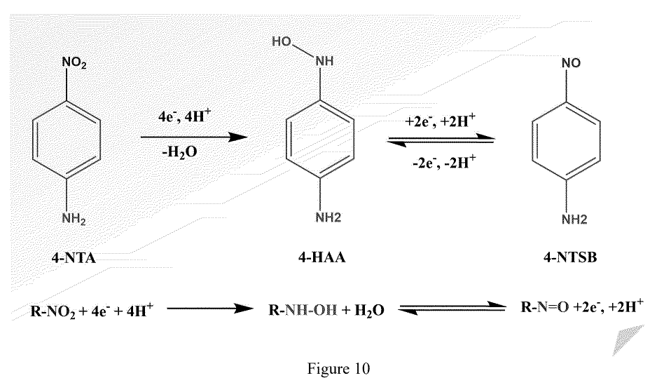

According to the present invention, the organic pollutants in a contaminated water sample include nitroanlines (NTA), such as 2-Nitroanline (2-NTA), 3-Nitroaniline (3-NTA), and 4-Nitroaniline (4-NTA), particularly 4-NTA. The structure of 4-NTA is shown in FIG. 10.

According to the invention, the composite material NC/rGO-A may be prepared by a method comprising steps of:
(i) dissolving niobium carbide and reduced graphene oxide aerogel in water as a mixture;
(ii) stirring to homogenize the mixture; and
(iii) sonicating the mixture thoroughly to produce the composite material NC/rGO-A.

According to the present invention also provides a NC/rGO-A based modified electrode, called as NC/rGO-A/SPCE that has great electrical conductivity.

In the present invention, the NC/rGO-A/SPCE may be prepared by the method comprising the steps of
(i) rinsing a bare screen-printed carbon electrode in distilled water and ethanol to obtain a rinsed bare screen-printed carbon electrode;
(ii) coating the composite material NC/rGO-A onto the rinsed screen-printed carbon electrode obtained in step (i) to obtain a screen-printed carbon electrode with coating of NC/rGO-A; and
(iii) drying the screen-printed carbon electrode with coating of NC/rGO-A obtained in step (ii) electrode coated with NC/rGO-A.

According to the present invention, the NC/rGO-A/SPCE has the greatest scan rate analysis of the surface area values comparing to bare SPCE, NC/SPCE, and rGO-A/SPCE, suggesting that the NC/rGO-A/SPCE has a remarkable improvement on scanning ability.

In the invention, the NC/rGO-A/SPCE has the best cathodic peak current and peak potential shift near to potential about-0.67 V, suggesting that the NC/rGO-A/SPCE is the best suitable for the detection of 4-NTA when comparing to bare SPCE, NC/SPCE, and rGO-A/SPCE.

It is ascertained in the examples that the NC/rGO-A based modified electrode (NC/rGO-A/SPCE) has an excellent selectivity exposing to interfering compounds comprising 4-nitrophenol, aminophenol, nitrobenzene, acetaminophen, carbendazim, chlorine ions, sodium ions, glucose, mercury, and hydroquinone.

The NC/rGO-A/SPCE may be applied to detect and monitor 4-NTA in real sample using differential pulse voltammetry (DPV) method, comprising industrial waste water, river water, or lake water. The 2D nanostructured composite material NC/rGO-A incorporated for 4-NTA sensing has been effectively identified that the sensing ability for calculating recovery percentage of the real samples were excellent. It is more likely attributable to the nanohybrid combination that diminished the intersheet aggregation, improved surface area and high conductivity.

The present invention is further illustrated by the following examples, which are provided for the purpose of demonstration rather than limitation.

### EXAMPLES

### Material and Method

Reduced Graphene Oxide Aerogel (rGO-A) Synthesis

Thermal, chemical, photo-thermal, microbiological production, exfoliation, reduction method, and various other processes can all be employed to generate (reduced graphene oxide) rGO. Each of those techniques significantly diminishes levels of oxygen.

In the present invention, we employed Hummers' approach for synthesizing rGO. 1.5 g of graphite powder, 1.5 g of NaNO₃, and 4 g of KMnO₄ were continuously stirred together to form a mixture. The mixture was developed in 75 mL of highly concentrated H₂SO₄ and then heated under a cold bath for 3 hours at 50° C. under continuous stirring. The temperature at which it was heated was raised to 95° C. after adding 150 mL of distilled water (DW). Each of the following treatments has been carried out in a vigorous stirring condition. The solution of hydrogen peroxide was incorporated after 15 minutes and stirred for one hour. Additionally, the resulting solution was extracted and washed many times with 37% hydrochloric acid and DW, resulting in a pH value of 6.5-7.0 recorded. The resultant mixture was subsequently vacuum-dried over 24 hours, resulting in the formation of graphene oxide (Guerrero-Contreras and Caballero-Briones, 2015).

To prepare rGO aerogel, the resulting amount of graphene oxide (GO) has been further reduced following the as mentioned process as follows. 0.2 g of GO was ultrasonicated over 2 hours with double distilled (DD) water. To regulate PH-10, the solutions of sodium hydroxide and ascorbic acid were constantly added into the previous solution while sonication (Abdolhosseinzadeh et al., 2015). After the sonication process, it was transferred into a Teflon-lined autoclave and heated at 120° C. for 12 h. After allowing the product to cool at ambient condition, it was rinsed with DW and ethanol to eliminate unwanted reactants. Finally the obtained product was freeze-dried for 24 hours which resulted with the formation of rGO aerogel (Chen et al., 2013; Kokulnathan et al., 2021a; Lee et al., 2021; Nundy et al., 2021).

### Niobium Carbide (NC) Preparation

In a beaker, a mixture of 0.1 M niobium chloride and 0.3 M ammonium carbonate was stirred (500 rpm) for 2 hours to obtain NC. Followed by the completely dispersed solution, the mixture was placed in an autoclave over 13 hours at 130° C. The obtained solution was carried out for washing and drying which was then calcined for 9 hours at 900° C. in nitrogen atmosphere (Ma et al., 2012; Ma and Du, 2008; Medeiros et al., 2002; Shi et al., 2005).

### NC/rGO-A Preparation

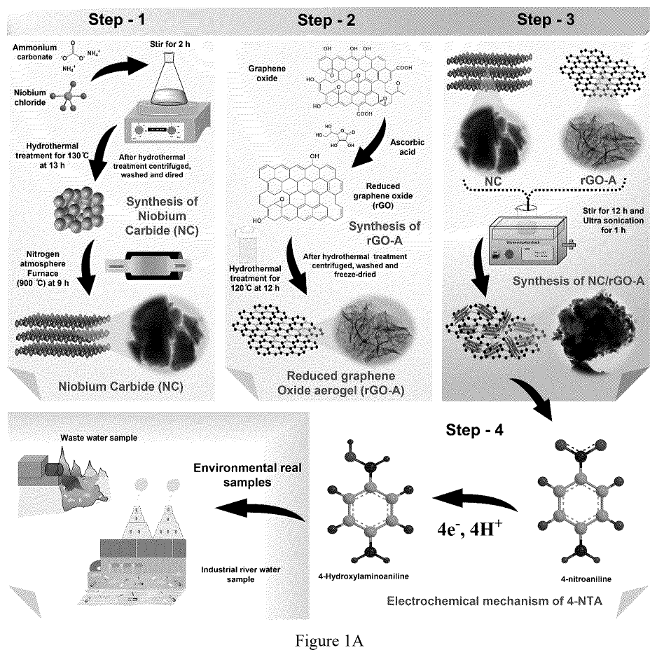

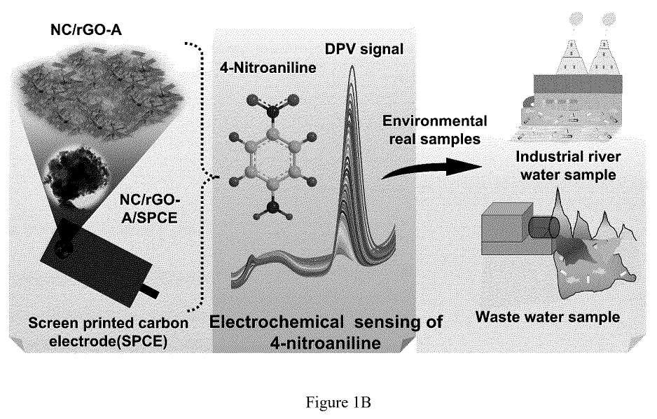

Further, a nanocomposite material NC/rGO-A was prepared by ultrasound assisted sonication procedure. The NC and rGO-A compounds were combined in a ratio of 1:1 before the dissolution in DI water, followed by stirring the both for 12-hours homogenizing and sonicating the both for 1-hour fabricating. During the 1-hour sonicating procedure, the NC and rGO-A were thoroughly sonicated to form NC/rGO-A. FIG. 1A shows a graphical illustration of the procedure for NC, rGO-A, NC/rGO-A synthesis approach, and electrochemical analysis for 4-NTA.

Electrode Modification of NC rGO-A

NC, rGO-A, NC/rGO-A, and bare screen-printed carbon electrode (SPCE) were developed via fabrication over the electrode surface. During fabrication, the SPCE surfaces were washed to eliminate the contaminants from earlier experiments. The SPCE was rinsed with distilled water and ethanol before being fabricated with other materials. The perfect amount of NC/rGO-A, around 4 μL, was modified by drop coating the materials onto the rinsed bare SPCE and dried out for a period of ten minutes in a hot air oven at 50° C. The SPCE was thus fabricated and 4-NTA detection was performed using a three-electrode system. FIG. 1B shows the application of NC/rGO-A based modified electrode (NC/rGO-A/SPCE) on analyzing differential pulse voltammetry (DPV) signal of 4-NTA existing in waste water or industrial river water samples.

### Results

### X-Ray Diffraction (XRD) Analysis

The crystal structure of niobium carbide (NC), reduced graphene oxide (rGO), and niobium carbide/reduced graphene oxide aerogel (NC/rGO-A) composite were analyzed by the powder XRD analysis.

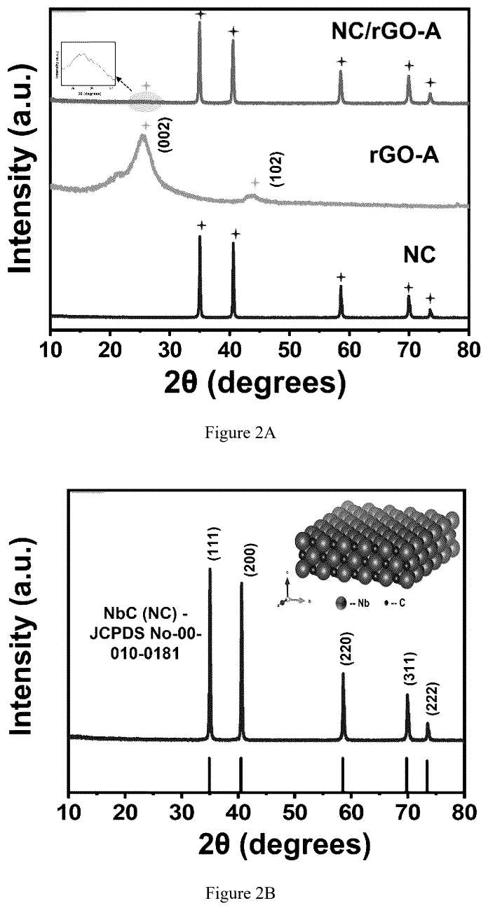

As shown in FIG. 2A, the XRD pattern of NC showed five diffraction peaks at 35.1°, 40.6°, 58.5°, 69.9°, and 73.5° corresponding to the (111), (200), (220), (311), and (222) hkl planes. The obtained result was well matched with the Joint Committee on Powder Diffraction Standards (JCPDS) card No-00-010-0181 and compared with the stick pattern of the accepted JCPDS card number as demonstrated in FIG. 2B of NC. The NC crystal system-cubic, space group-Fm-3m and space group number about-225. The crystal structure with space filling of NC was depicted with the values a=b=c=4.4699 Å and alpha=beta=gamma=90° represented as inset in FIG. 2B. The reduced graphene oxide aerogel (rGO-A) was observed with the broad diffraction peak at around 25.4° and 43.5° corresponding to the (002) and (102) hkl planes which can be ascribed to multiple-layer rGO-A nanosheets. The successful formation of NC/rGO-A were observed with reduced intensity of rGO-A which can be seen as an enlarged inset. Thus, it is ascertained that the NC/rGO-A contained no impurity peaks. For the as prepared samples, we have measured average crystalline size, micro strain and dislocation density. The samples were calculated with the as determined formula as Scherrer's equation

$$D = \frac{K λ}{β Cos θ} ,$$

the micro strain equation

$$ε = \frac{β}{4 tan θ} ,$$

and the dislocation density equation as

$$δ = \frac{1}{{D}^{2}} .$$

The illustrated parameters are D, and K, indicating the Scherrer constant (0.94), the full-width half maxima of the high-intensity plane, the X-ray wavelength (1.54), and the diffraction angle. All the above said values were determined and given in Table 1 representing the average crystalline size, strain, and dislocation density of the synthesized materials (NC and rGO-A).

TABLE 1
The (average) crystalline size, micro strain, and dislocation
density of all the prepared compounds NC, and rGO-A.

| | | Full width | | | |
|---|---|---|---|---|---|
| Prepared | | half | Crystal size | Micro strain | Dislocation |
| materials | hkl planes | maximum | (nm) | (×10⁻³) | density δ |
| NC | (111) | 0.1248 | 66.7 | 1.725 | 0.224 |
| | (200) | 0.1716 | 49.3 | 2.024 | 0.410 |
| | (220) | 0.1404 | 64.8 | 1.091 | 0.237 |
| | (311) | 0.1308 | 74.1 | 0.816 | 0.182 |
| | (222) | 0.2184 | 45.3 | 1.276 | 0.485 |
| NC - Average crystal size, micro strain, | | | 60.04 | 1.38 | 0.30 |
| dislocation density | | | | | |
| rGO-A | (002) | 0.2376 | 34.2 | 4.58 | 0.851 |
| | (102) | 0.1687 | 50.7 | 1.84 | 0.388 |

| | | | |
|---|---|---|---|
| rGO-A - Average crystal size, micro strain, | 42.45 | 3.21 | 0.61 |
| dislocation density | | | |

### Raman and FTIR Studies

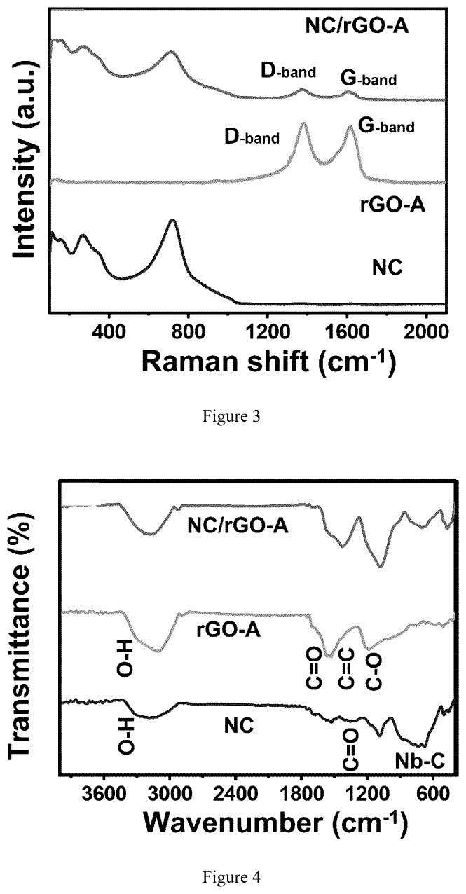

As shown in FIG. 3, the NC, rGO-A, and NC/rGO-A were examined further with Raman spectroscopy technique.

The Raman spectrum of NC was raised with the vibrations at 164, 267, 716, 911 cm⁻¹, those correspond to the internal Nb—C vibrational modes (Santhan and Hwa, 2022b; Wang et al., 2021). The Raman spectrum illustrated the presence of rGO-A with D band (1381 cm⁻¹) representing the lattice disorder, while the G band (1615 cm⁻¹) represents sp²-hybridized carbon groups. The peak intensity ratio (I_{D}/I_{G}) of rGO-A was determined about 0.85 (Santhan et al., 2022). The aforementioned NC and rGO-A peaks were all together corresponding to the NC/rGO-A presence. The I_{D}/I_{G} ratio in the NC/rGO-A (0.87) was smaller than the ratio of rGO-A, revealing that the NC/rGO-A exhibited a larger number of defects on the surface thereof. Also, it was able to prove that NC/rGO-A was presented with the absence of other additional relevant peaks in our investigation.

### Fourier Transform Infrared (FTIR) Spectrum Analysis

The functional group presence of the prepared combination of samples were identified with FTIR spectrum as analyzed. As shown in FIG. 4, the FTIR spectrum of NC, rGO-A, and NC/rGO-A obtained at the corresponding wavenumbers.

As a result, the NC was associated with vibration around 732, 1094, 1537, and 3200 cm⁻¹. The O—H vibration associated with stretching has been identified for the wider peak determined at 3225.4 cm⁻¹. The absorbed molecules of water were responsible for the vibration at 1678 cm⁻¹. Other peak values at 826 and 1516 cm⁻¹ has been assigned to Nb—C bond in the material, whereas another 1171 cm⁻¹ vibration is ascribed to C═O (Mahle et al., 2022) (Santhan and Hwa, 2023, 2022a).

The result of FTIR spectrum indicated the rGO-A spectrum was associated with a broad spectrum at 3150 cm⁻¹, corresponding to strong stretching mode of O—H band. An absorbance peak around 1548 cm⁻¹ was raised because of the C═C stretching mode; as well as peaks at 1705, 1172, and 1038 cm⁻¹, were related to the stretching modes of C═O, C—OH, and C—O, accordingly (Gong et al., 2015; Li et al., 2020).

NC/rGO-A was well proven from the Raman study with the presence of both the rGO-A and NC bonds. As a result, it can be connected the most relevant wavelengths with their corresponding functional categories. NC and rGO-A materials were effectively integrated to produce the NC/rGO-A with all of the corresponding functional categories.

### X-ray Photoelectron Spectroscopy (XPS)

The construction of NC/rGO-A was examined with XPS analysis.

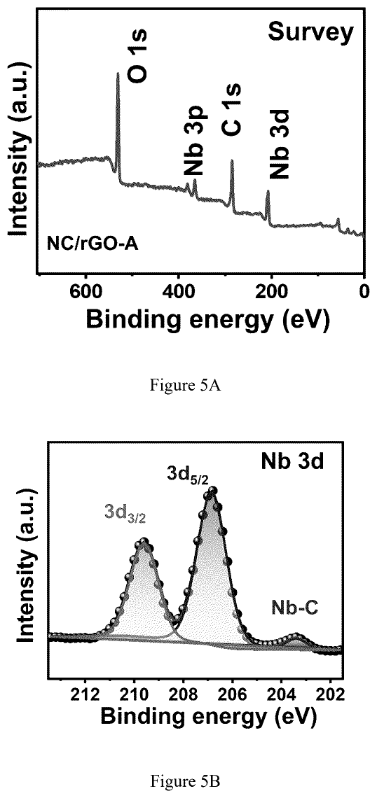

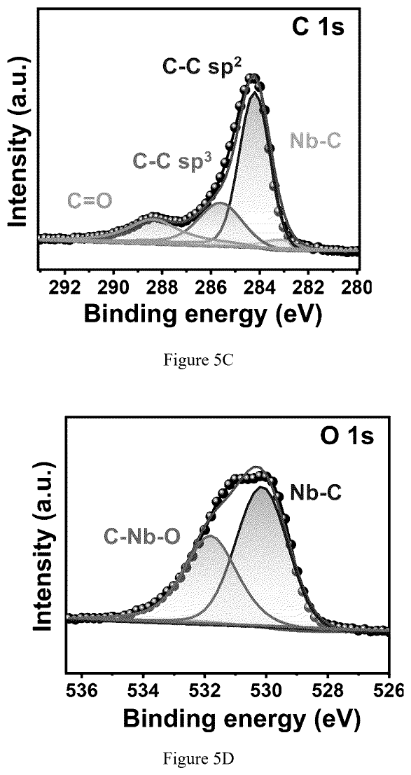

As shown in FIG. 5A, the overall XPS survey spectra of NC/rGO-A, which represented the elemental presence as Nb 3d, C 1s, and O 1s.

As shown in FIG. 5B, the presence of Nb 3d was deconvoluted into three different binding energies (BEs) at 206.8, 203.3, and 209.5 eV. These binding energies are accredited to three spin-orbital splitting of Nb—C, Nb 3d_{5/2} and Nb 3d_{3/2} respectively (Santhan and Hwa, 2022a) (Liu et al., 2020) (Pg et al., 1998).

As shown in FIG. 5C, the C Is was fitted with four peaks at 282.8, 284.1, 285.6, and 288.6 eV. The four peaks were ascribed to four spin-orbital splitting of Nb—C, C—C(sp²), C—C(sp³), and C—O, which confirmed the presence of C 1s (Gupta et al., 2018).

As shown in FIG. 5D, oxygen 1s spectrum of NC/rGO-A was deconvoluted into two peaks at binding energy of 530.1 and 531.8 eV, which is ascribed to the Nb—C and C—Nb—O (Pg et al., 1998). The obtained elemental composition has confirmed the successful formation of NC/rGO-A.

### Morphological Investigation

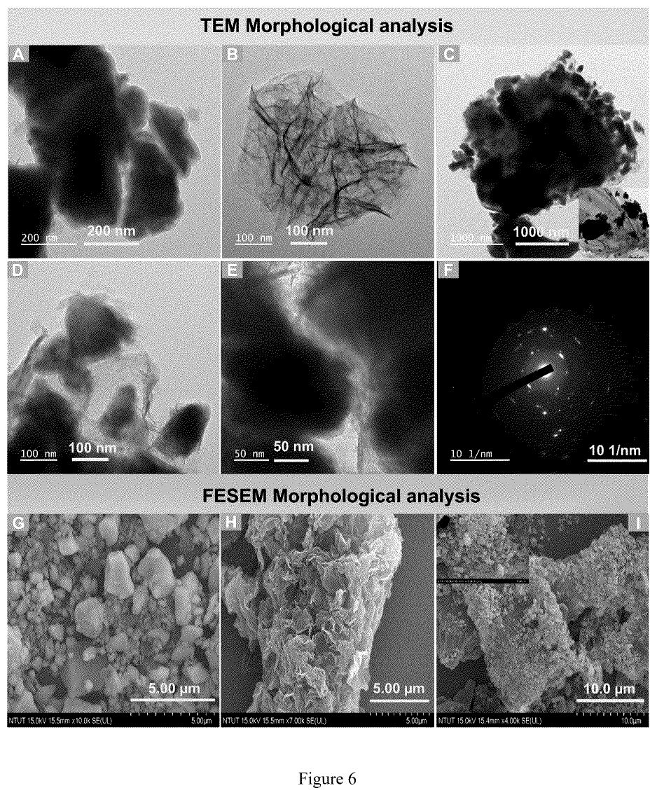

Transmission electron microscope (TEM) and Field emission scanning electron microscope (FESEM) studies were performed to investigate the morphological arrangements of NC, rGO-A, and NC/rGO-A. The TEM images of NC, rGO-A, and NC/rGO-A were depicted in picture A-E of FIG. 6; while the FESEM images of NC, rGO-A, and NC/rGO-A were depicted in picture G-I of FIG. 6; the selected area electron diffraction (SAED) pattern of NC/rGO-A was shown in picture F of FIG. 6.

As shown in FIG. 6: picture A shows a bulk nanosheet-like structure which was associated to the NC. This presented an uneven structure, and rough surfaces, and also was aggregated with other sheets. Although the bulk nanosheet-like structure forms a nanostructured material layer, it quickly connected and then collapsed, leading to generation of a rock-like material structure. Picture B purely represented the rGO-A nanosheets, indicating more wrinkles and folds, suggesting that might allow stacking and aggregation between the single rGO-A nanosheets that were formed by significant π-π interaction. Picture C-E demonstrated that the rGO-A layers were distributed evenly containing a significant quantity of NC. This phenomenon validates the development of an NC nanostructure that is equally wrapped around rGO-A wrinkled nanosheets. The uneven dark bulky stone like structure were demonstrated as NC and lighter nanosheet like structure with thin layers were associated to rGO-A. The TEM images of NC, rGO-A, and NC/rGO-A were observed with magnification about 200, 100, and (1000, 100, 50) nm.

The FESEM images of the synthesized samples including NC, rGO-A, and NC/rGO-A are shown in picture G-I of FIG. 6. Picture G shows NC nanostructured sheets that were very thick and flattened, and distributed unevenly. The NC nanostructured sheets had a structure that was uneven and rough outer surfaces, as it also came aggregated with other nanostructures. Picture H shows a wrinkled sheet-like structure of rGO-A layers with dimensions ranging within the μm range. The hydroxy and epoxy structures were assigned arbitrarily along the rGO-A's base surface or edge. Developing various bonds enabled rGO-A in the material to bind with numerous organic or inorganic materials. Picture I shows the NC/rGO-A in which NC nanostructures were anchored and stacked with rGO-A wrinkled sheets, illustrating the successful result of pulverizing and ultrasonic treatment. As evidenced using surface studies like TEM, and FESEM, it has been shown that NC has properly anchored into rGO-A sheets with the absence of other additional contaminants, leading to a success of the composite with a hybrid structure.

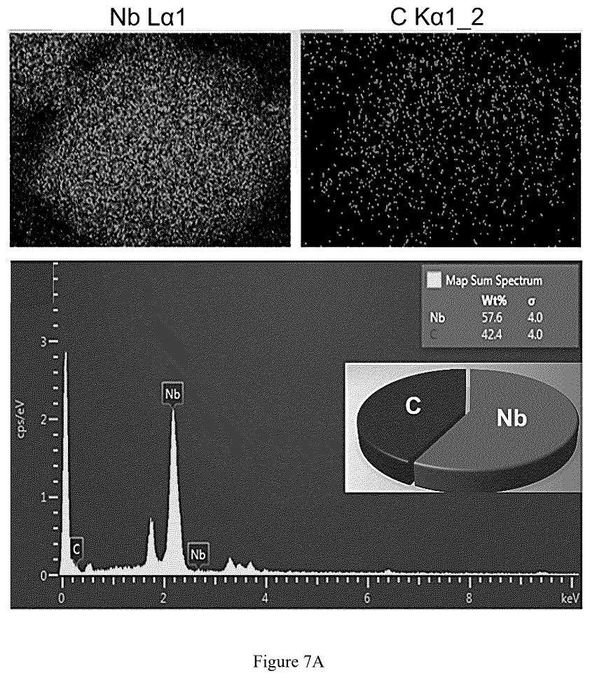

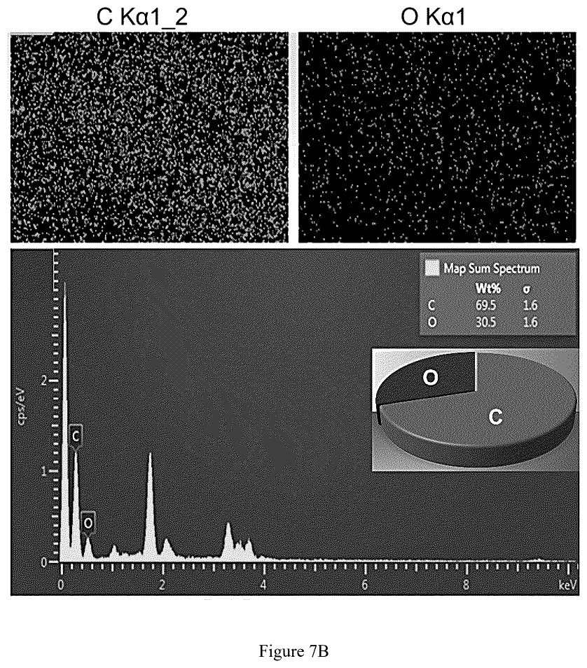

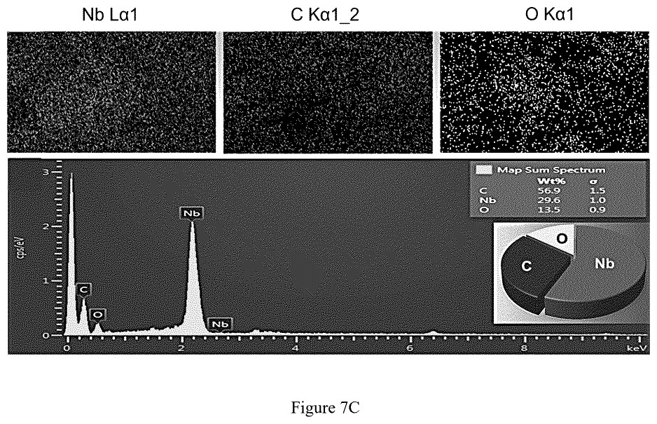

Furthermore, the composition of each element was determined by precise elemental mapping with the Energy Dispersive X-Ray Spectroscopy (EDX) assisted with FESEM investigation, which demonstrates the elemental percentage. Thus, the EDX analysis also proved the existence of the compounds as Nb, C, and O with no other presence of any possible unrelated contaminants. FIG. 7A shows the elemental mapping and EDX spectrum of NC, revealing that it contained 57.6% niobium (Nb) and 42.4% carbon (C). FIG. 7B shows the elemental mapping and EDX spectrum of rGO-A, revealing that it contained 69.5% C and 30.5% oxygen (O). FIG. 7C shows the elemental mapping and EDX spectrum of NC/rGO-A, revealing that it contained 29.6% Nb, 56.9% C, and 13.5% O.

### Electrochemical Resistance and Surface Area Analysis

The electrochemical impedance spectroscopy (EIS) was examined for the unmodified and different materials modified electrodes such as bare screen-printed carbon electrode (SPCE), NC/SPCE, rGO-A/SPCE, NC/rGO-A/SPCE as scrutinized to analyze the charge transfer resistance (R_{ct}). The electrolyte solution as 5 mM [Fe(CN)₆]^{3-/4-} and 0.1 M of KCl were used with the three-electrode setup.

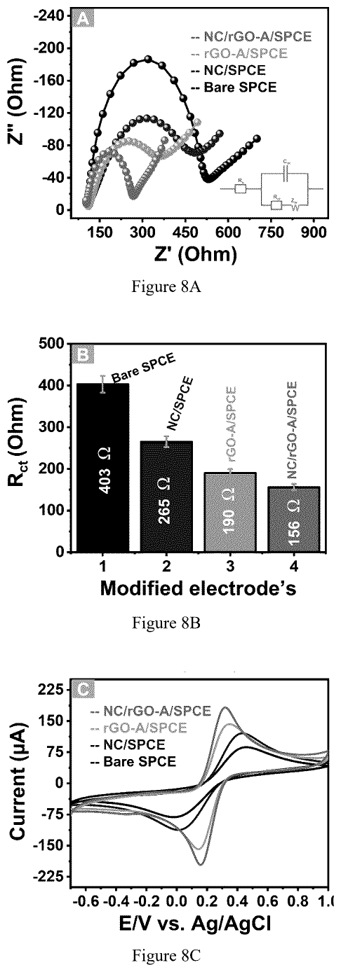

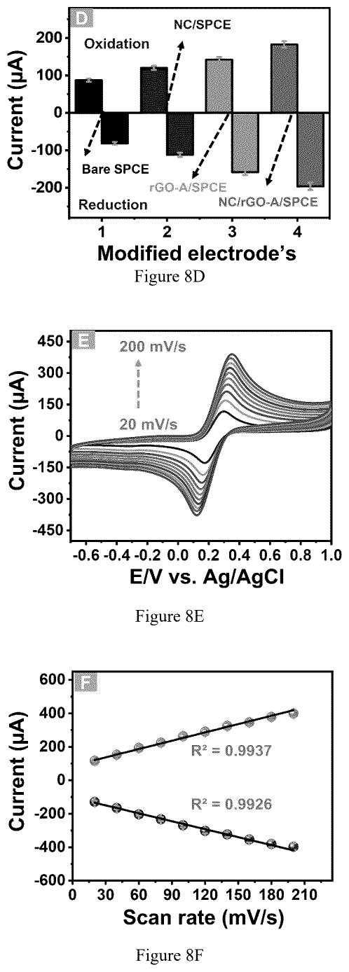

As shown in FIG. 8A, the EIS Nyquist plot for modified and unmodified electrodes and the Randles circuit image were presented.

As shown in FIG. 8B, the results of Randles circuit illustrated several significant interfacial properties notably (R_{ct}) charge transfer resistance, (C_{dl}) double layer capacitance, (Rₛ) solution resistance, and (Z_{w}) the Warburg constant. All modified and unmodified electrodes were analyzed with the fixed frequency conditions from 1 Hz to 100 kHz. Furthermore, the Nyquist plots with high and low frequency signals corresponded to the electron transfer kinetics and mass transfer owing to the impedance. Overall, the Ret value in the Nyquist diagram was directly proportional to the diameter of the semicircle. From the Nyquist results, NC/rGO-A-modified SPCE (NC/rGO-A/SPCE) displayed a relatively tiny semicircle signal due to its lower R_{ct} of 156Ω, which is smaller than rGO-modified SPCE (rGO-A/SPCE) (190Ω), NC-modified SPCE (NC/SPCE) (265Ω) and bare screen-printed carbon (403Ω) electrodes. At the NC/rGO-A/SPCE, a lower resistance correlated to higher electron transferred with enhanced electrical conductivity. The extensively present active areas exhibited by the interaction of both NC and rGO-A interfaces determined this outstanding conducting performance. In addition, NC/rGO-A/SPCE is worthy of detecting compounds since it has greater conductivity. Further, the cyclic voltammetry (CV) approach was employed to calculate the peak-to-peak potential difference (ΔEp) and active surface area of bare SPCE, NC/SPCE, and rGO/SPCE, and NC/rGO-A/SPCE based on the redox reaction activity.

As shown in FIG. 8C, the CV analysis of modified SPCE along with the unmodified SPCE were presented. The fixed potential was-0.7 to 1.0 V and scan rate kept at 50 m V/s and the 5 mM [Fe(CN)₆]^{3-/4-} with 0.1 M of KCl electrolyte solution were employed for all the electrodes to be studied. All those electrodes examined for CV displayed a clearly defined redox curve. In the beginning, the surface of the bare SPCE presented a redox peak with a widened and expanded peak with redox current response and potentials at Ipa—87.23 μA, Ipc—−81.62 μA and Epa—0.456V, Epc—−0.02 V. The NC/SPCE (Ipa—120.1 μA, Ipc—−112.3 μA and Epa—0.434V, Epc—0.008V) and rGO-A/SPCE (Ipa—142.2 μA, Ipc—−158.5 μA and Epa—0.348V, Epc—0.141V) had a greater redox peak with reduced peak-to-peak distance and finer peaks compared to bare SPCE. All the bare SPCE, NC/SPCE, and rGO-A/SPCE were compared, while the NC/rGO-A/SPCE (Ipa—182.5 μA, Ipc—196.6 μA and Epa—0.320V, Epc—0.157V) had a greater peak current than all other peaks with a smaller peak distance. The histogram of the above analysis of the different electrodes' performance is shown in FIG. 8D.

As shown in FIG. 8E, the scan rate analysis of the surface area values and the electrolyte solution were performed. The electrochemical conditions were the same as studied with the above analysis with the only change with the different scan rates like 20 to 200 m V/s for the modified or unmodified electrodes. The Randles-Sevcik formula can be used for determining the electrochemically active surface area of all modified electrodes. The derivation is as described below (Elgrishi et al., 2018):

$${I}_{p} = ( 2.69 \times {10}^{5} ) {n}^{3 / 2} {AD}^{1 / 2} C {υ}^{1 / 2}$$

wherein the “Iₚ” refers to peak current, the “n” refers to the number of electrons transferred, the “A” refers to electroactive surface area, the “D” refers to diffusion coefficient, the “C” refers to concentration of electrolyte solution, and the “ν” refers to potential scan rate.

As shown in FIG. 8F, the NC/rGO-A/SPCE linear plot of scan rate vs current was performed, in which the linear equations were Ipa=1.5801x+95.12 with respect to the correlated coefficient R²=0.9937 and Ipc=−1.5094x−110.07 with the results of the correlation coefficients R²=0.9926. Considering the aforementioned equations, the calculated surface areas for bare SPCE, NC/SPCE, rGO-A/SPCE, and NC/rGO-A/SPCE were respectively 0.034, 0.051, 0.078, and 0.104 cm². The greatest area of the surface was measured with NC/rGO-A/SPCE. Therefore, this newly improved characteristics is most likely the result of the integration of the NC/rGO-A/SPCE.

### Electrochemical Detection of 4-NTA

The electrochemical detection of 4-NTA was examined at bare SPCE, NC/SPCE, rGO-A/SPCE, NC/rGO-A/SPCE firstly to analyze their performances.

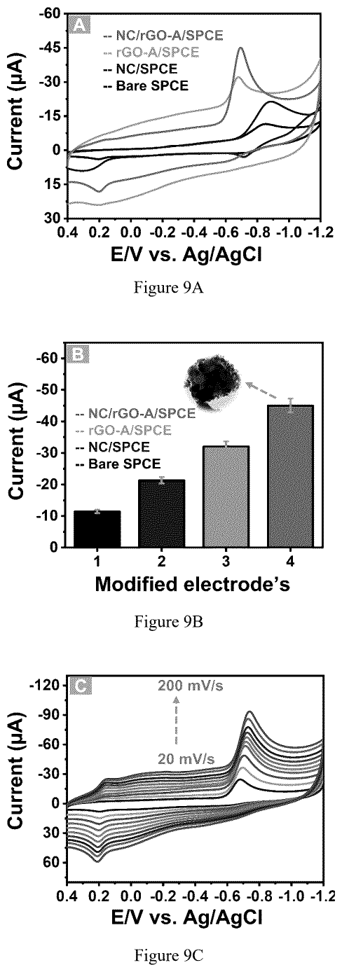

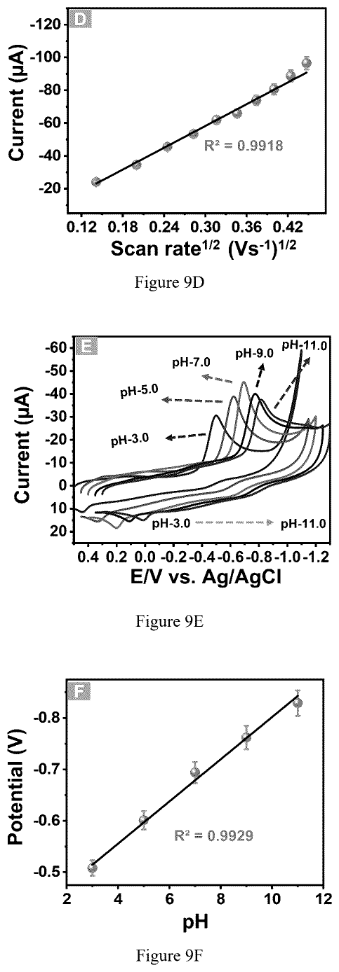

FIG. 9A shows the different film CV plots of bare SPCE, NC/SPCE, rGO-A/SPCE, NC/rGO-A/SPCE. The experimental parameters were as studied was in 0.1 M of PBS pH 7.0 and 50 m V/s of constant scan rate with 100 μM of 4-NTA, at the fixed potential of 0.4 V to −1.2 V. The bare SPCE showed a cathodic peak current about (−11.45 μA), NC/SPCE—(−21.32 μA), rGO-A/SPCE—(−32.08 μA), and NC/rGO-A/SPCE—(−45.04 μA). FIG. 9B represents histogram graph of the tested SPCE. The bare SPCE was able to respond with lesser reduction current since no enough reactants to contribute its reaction with 4-NTA reduction. In comparison with all other modified SPCE, the NC/rGO-A/SPCE has the best cathodic peak current and peak potential shift near to potential about −0.67 V. It is ascertained that the NC/rGO-A/SPCE is the best suitable for the detection of 4-NTA.

During the electrochemical sensing of 4-NTA, different scan rate analysis was also performed. The scan rate was varied from 20 to 200 mV/s with 100 μM 4-NTA addition to the NC/rGO-A/SPCE. 0.1 M phosphate buffered saline (PBS) (pH 7.0) was used as electrolyte at the potential window as 0.4 V to −1.2 V.

As shown in FIG. 9C, the CV curves of different scan rate were performed. When sweep rate increased, the current response simultaneously increased with no more negative potential shift during the study. FIG. 9D shows the linear plot of square root of different scan rate vs current and the linear regression equation for the study was Ipc=232.99x−11.572 and R²=0.9918. The faster reaction was observed due to the weakly reacted ions which were not involved in the response at the electrode/electrolyte interactions. FIG. 10 shows the electrochemical reduction mechanism of 4-nitroaniline to 4-hydroxylaminoaniline to form nitrosobenzene with 4 electron/proton transfer process at the NC/rGO-A/SPCE (Manavalan et al., 2019; Yamuna et al., 2021a). With the reduction reaction peak at −0.69 V, the reduction to 4-hydroxylaminoaniline can be well evaluated.

The PBS was selected with a suitable pH level for each investigation of 4-NTA at NC/rGO-A/SPCE. With the presence of 4-NTA (100 μM) addition, the sweep rate was kept at 50 mV/s for different pH range of 3.0, 5.0, 7.0, 9.0, and 11.0. The potential window was changed (0.5 V to −1.3 V).

As shown in FIG. 9E, a reduction peak was observed at a potential of −0.50 V, with a cathodic current of 30.56 μA at pH 3.0. When the pH was adjusted to 5.0, the potential shifted, and a higher reduction peak current was obtained. As the pH was gradually increased, the response improved, reaching a higher level at pH 7.0 with a potential of −0.69 V, resulting in a better reduction peak current of 45.1 μA. This enhanced response was utilized in subsequent studies for 4-NTA sensing. Even though pH 9.0 was examined, the reaction diminished and stayed to be decreased at pH 11.0.

As shown in FIG. 9F, the linear regression equation for various pH vs potential was observed to be Ipc=−0.0402x−0.3978 and R²=0.9929. According to the linear equation, the value 59 m V is nearly equivalent to the Nernst value (Elgrishi et al., 2018). Therefore, the electrode/electrolyte interaction distributes an equal number of electrons.

### Different Concentrations Analysis

The different concentration analysis at NC/rGO-A/SPCE was studied to better understand its response at lower and higher concentration of 4-NTA. The experimental conditions as 0.1 M PBS (pH 7.0), 50 mV/s scan rate which was kept constant, and then the potential window was fixed from 0.4 V to −1.2 V.

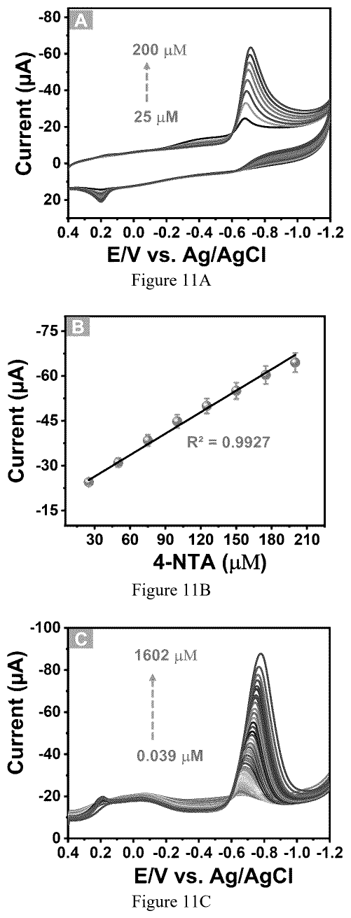

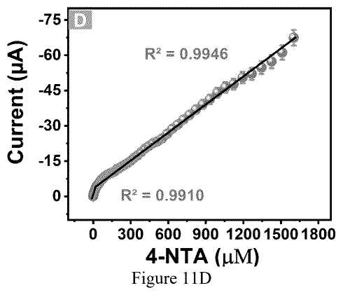

As shown in FIG. 11A, the CV curves showed response for varied concentrations from 25 μM to 200 μM. The reduction current response was higher with the 4-NTA higher concentrations. As the result, there was no reduction or change toward a negative potential at the NC/rGO-A/SPCE. The outcomes strongly indicated the NC/rGO-A/SPCE's excellent sensing performance.

As shown in FIG. 11B, the linear plot for varied concentration vs current and the linear results identified corresponded to the linear equations observed to be Ipc=0.2291x+20.314 with the correlation coefficient about R²=0.9927.

As shown in FIG. 11C, the DPV studies at NC/rGO-A/SPCE was analyzed for utmost specific detection of 4-NTA. Following the condition of 0.1 M PBS at pH 7.0 and potential window up to 0.4 V to −1.2 V, the 4-NTA level was efficiently injected repeatedly between the linear range from lower concentrations 0.039 μM to higher concentrations 1602 μM. The DPV profiles observed at NC/rGO-A/SPCE certainly showed linearly higher reduction peaks at about (−0.69 to 0.73 V). A consecutive 4-NTA injection resulted in a consistent rise in current responsiveness with stronger reduction levels. The excessive volume of 4-NTA molecules presented on the NC/rGO-A/SPCE interface generated a minor change in the DPV profiles.

As shown in FIG. 11D, a regression formula was applied to calibrate the continuously increased 4-NTA injection into two distinct linear intervals. The calibration values are Ipc=0.9789x+0.0568 and R²=0.9910 at lower concentration and Ipc=0.0399x+2.9849 and R²=0.9946 at higher concentration of 4-NTA. The limit of detection (LOD) and sensitivity can be measured by examining the as-determined DPV graphs employing the following equation. The limit of detection LOD=3SD/σ, the standard deviation obtained for the performed analysis is SD and the slope value of the calibration plot σ (Nataraj and Chen, 2021; Santhan et al., 2022). The LOD 4.6 nM for 4-NTA sensing is determined by employing the graphs generated in the case of DPV, whereas the NC/rGO-A/SPCE sensitivity has been determined to be 27.96 μA μM⁻¹ cm².

As shown in Table 2 below, the comparison of the performance of the modified SPCE against previous 4-NTA sensing results were presented. According to the table, the combination of NC with rGO-A enhanced the electrocatalytic efficiency at the NC/rGO-A/SPCE surface, leading to more efficient 4-NTA sensing.

Each abbreviation of the Table 2 can be interpreted as follows: NC/rGO-A/SPCE-Niobium carbide/reduced graphene oxide aerogel/screen printed carbon electrode, CV-Cyclic voltammetry, DPV-Differential pulse voltammetry, LC-AD-liquid chromatography with amperometry detection, IT-Amperometry, ¹CuNPs-CH-Copper nanoparticles embedded chitosan, ²GCE-glassy carbon electrode, ³Chitosan-Ag NPs-Chitosan-silver nanoparticles, 4Ag-CPE-silver particles-carbon-paste electrode, ⁵BVG@C-g—C₃N₄@BiVO₄/Ag₂CO₃, ⁶CS@CPE-carbon paste electrode modified with a chitosan solution gelled in acetic acid, ⁷DTD/Ag-CPE-(6,7,9,10,17,18,19,20,21,22-decahydrodibenzo[h,r][1,4,7,11,15] trioxadiazacyclonanodecine-16,23-dione, DTD)-Ag nanoparticles (AgNP) modified carbon paste electrode (DTD/Ag-CPE) is fabricated, ⁹PC₉₀₀/GCE-porous carbon/glassy carbon electrode, and ¹⁰CME—chemically modified electrode.

### Interference Analysis

The NC/rGO-A/SPCE when subjected to several investigations, showed all of the vital characteristics and resulted in satisfactory results. However, the selectivity of the developed electrode is a significant sensing feature owing to its distinctive characteristics. The selectivity investigation was carried out in the presence of numerous substances classified as interfering, anti-interfering, and closely related family. Each of the previously mentioned substances was tested employing a similar electrochemical system in the DPV approach.

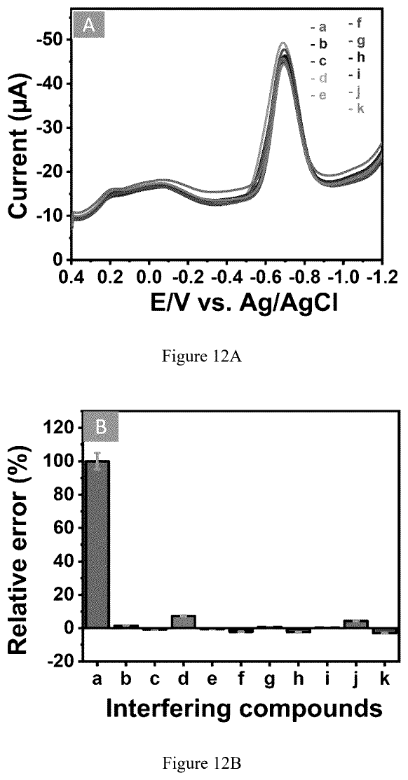

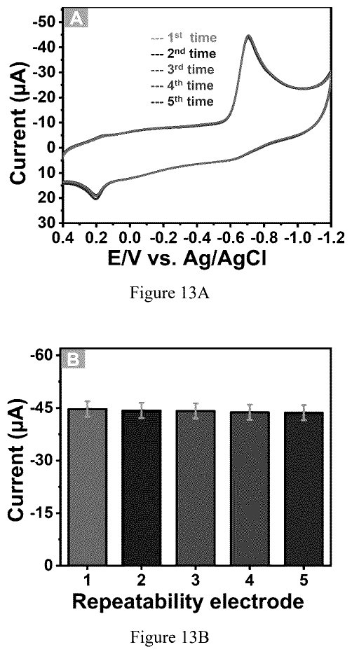

As shown in FIG. 12A, the DPV curves were provided when performing the selectivity examination involving each of the interfering compounds specified below (Manavalan et al., 2019; Nataraj et al., 2022; Yamuna et al., 2021b). The interfering compounds such as (b) 4-nitrophenol, (c) aminophenol, (d) nitrobenzene, (e) acetaminophen, (f) carbendazim, (g) chlorine ions, (h) sodium ions, (i) glucose, (j) mercury, and (k) hydroquinone were frequently supplied at a level that was twenty times greater than (a) 4-NTA. The obtained data clearly demonstrated the excellent selectivity exhibited by NC/rGO-A/SPCE at a lower level than the remaining exposed compounds. The primary impact of potential is a significant factor in the above investigation.

As shown in FIG. 12B, the relative error plot for the selectivity analysis were performed. Likewise, the NC/rGO-A/SPCE was found to be more effective with most of the characteristics that suit the electrochemical analysis of 4-NTA with outstanding performance.

Different Sensing Parameters at NC rGO-A SPCE

Analyses have been performed for crucial factors such as the electrode's “repeatability” (the ability to perform repeated monitoring), “reproducibility” (the capacity to recreate an electrode in other electrodes), and “stability” (the ability to maintain stable response for long time).

The repeatability had been evaluated using almost five consecutive studies over 4-NTA detection at NC/rGO-A/SPCE.

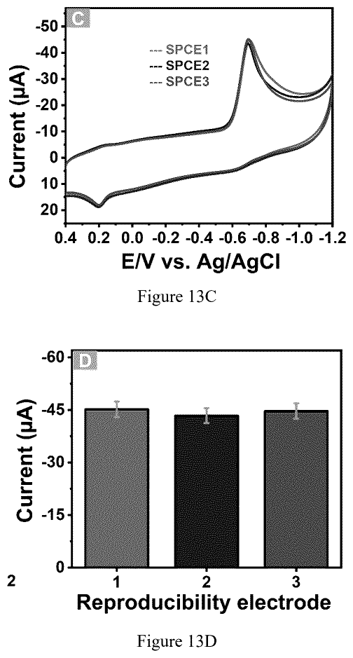

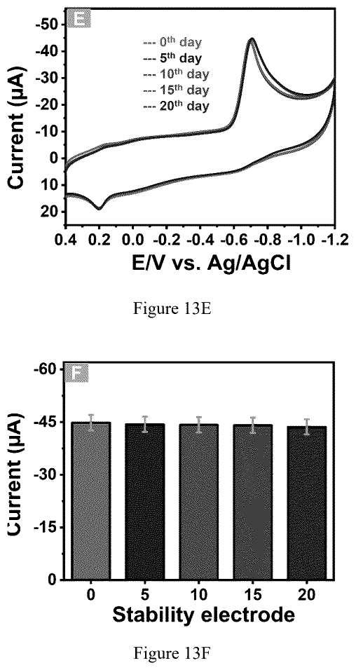

As shown in FIGS. 13A and 13B, the CV profiles were recorded in the repeatability investigation following the injection of (100 μM) 4-NTA at 50 mV/s scan rate. The outcomes illustrated linear results across all of the repeated tests, demonstrating the outstanding repeatability of the NC/rGO-A/SPCE.

As shown in FIGS. 13C and 13D, CV graphs were employed to measure the reproducibility of three separate electrodes. The aforesaid investigation employing the same conditions used in repeatability experiments. Considering only tiny differences, the reduction of 4-NTA was similar in each of the three SPCE. The outcomes illustrated similar results across all of the individual SPCE, demonstrating the outstanding reproducibility of the NC/rGO-A/SPCE.

As shown in FIGS. 13E and 13F, the stability study was displayed for a duration of twenty consecutive days with a 5-days gap until the twentieth days. CV was used as with similar experimental design as used during the repeatability investigation. The last day of measurements indicated a gradual decrease in reaction, however not higher in huge amount. This developed modification was attributable to the atmospheric conditions that existed throughout the preservation of the developed electrode following a period of stability testing. Therefore, all three investigations indicated that the NC/rGO-A/SPCE is better suitable for detecting 4-NTA with excellent performance.

### Real Sample Study

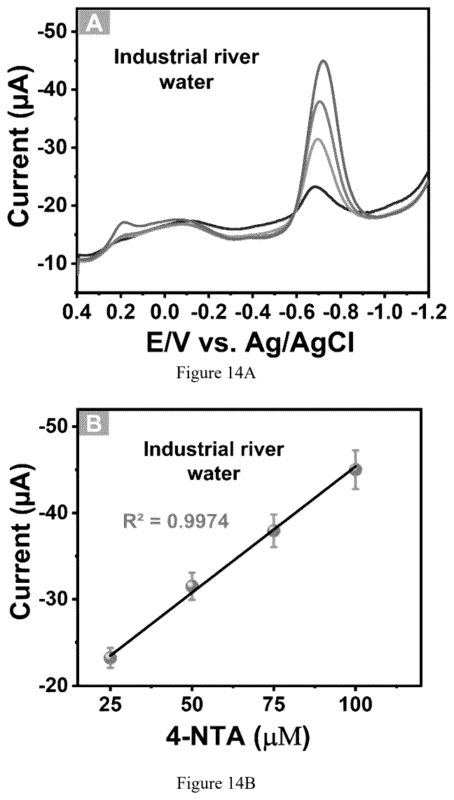

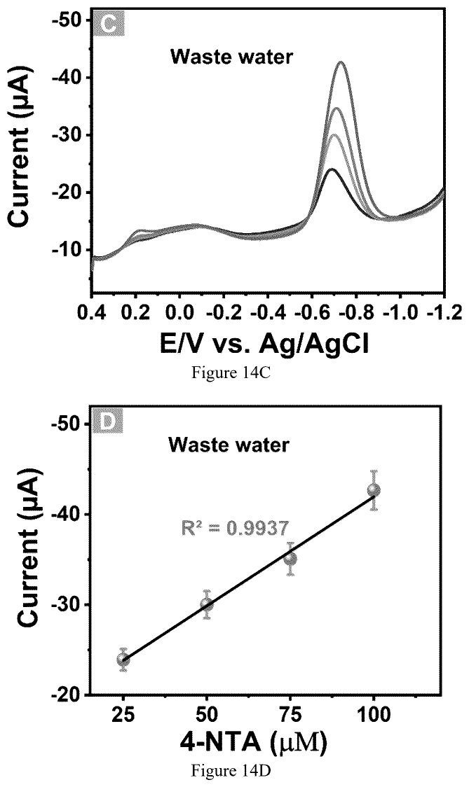

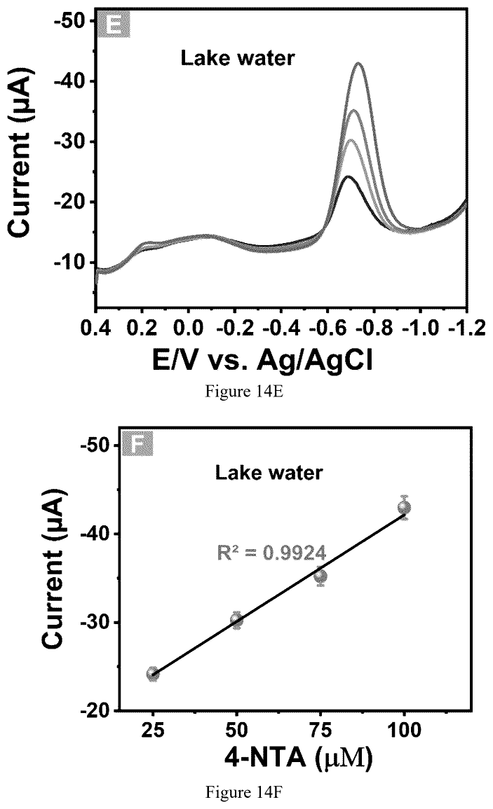

The NC/rGO-A/SPCE was further tested on real samples for monitoring of 4-NTA. Real samples of industrial river water, wastewater, and lake water had been collected and the 4-NTA was determined to be more significant (Nataraj et al., 2022; Yamuna et al., 2021b). Before conducting DPV experiments, the obtained real samples were pretreated. The centrifuged industrial river water, wastewater, and lake water were mixed with PBS. Each diluted sample was taken for DPV testing and studied with the NC/rGO-A/SPCE following the standard addition approach with 4-NTA being spiked ranging from 25-100 μM. As shown in FIGS. 14A, 14C, and 14E, DPV curves of industrial river water, waste water, and lake water were provided for the investigation, respectively; and the regression formulas were provided with the corresponding correlation coefficient given below: industrial River water Ipc=0.2873x+16.455 and R²=0.9974 (FIG. 14B); wastewater Ipc=0.2454x+17.58 and R²=0.9937 (FIG. 14D); and lake water Ipc=0.2456x+17.80 and R²=0.9924 (FIG. 14F). The recovery percentage of the real samples were calculated and tabulated in Table 3 below. The obtained data demonstrated the excellent sensing of 4-NTA at the NC/rGO-A/SPCE surface with high rates of significant recovery.

TABLE 3
Real sample analysis recovery percentage tabulation (n = 3).

| | | Detected | | |
|---|---|---|---|---|
| | Added | (μM) | | Detection Rate (%) |
| Sample | (μM) | DPV | HPLC | (Mean ± RSD) (n = 3) |
| Industrial river | 0 | — | — | — |
| water | 25 | 24.89 | 24.76 | 99.56 ± 0.13 |
| | 50 | 49.65 | 49.59 | 99.30 ± 0.06 |
| | 75 | 74.91 | 74.83 | 99.88 ± 0.08 |
| | 100 | 99.78 | 99.74 | 99.78 ± 0.04 |
| Waste water | 0 | — | — | — |
| | 25 | 24.94 | 24.88 | 99.76 ± 0.06 |
| | 50 | 49.51 | 49.39 | 99.02 ± 0.12 |
| | 75 | 74.83 | 74.76 | 99.77 ± 0.07 |
| | 100 | 99.04 | 98.89 | 99.04 ± 0.15 |
| Lake water | 0 | — | — | — |
| | 25 | 24.71 | 24.57 | 98.86 ± 0.14 |
| | 50 | 49.38 | 49.29 | 99.95 ± 0.09 |
| | 75 | 74.10 | 73.96 | 99.72 ± 0.14 |
| | 100 | 98.94 | 98.79 | 99.72 ± 0.15 |

The 2D nanostructured NC/rGO-A incorporated for 4-NTA sensing has been effectively identified and the outcomes were excellent. It is more likely attributable to the nanohybrid combination that diminished the intersheet aggregation, improved surface area and high conductivity. As a result of the existence of structural defects in rGO-A, greater active regions for effective transfer of electrons while coupled with NC have been successfully enhanced. The repeatability, reproducibility, and stability investigations indicated a stable performance under various conditions. The NC/rGO-A/SPCE has demonstrated outstanding detection ability with interference and stability investigations. The developed material of the present invention has a wide range of potential uses in real time detection with features to modify into a device fabrication.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

### REFERENCES

Abdolhosseinzadeh, S., Asgharzadeh, H., Kim, H. S., 2015. Fast and fully-scalable synthesis of reduced graphene oxide. Sci. Rep. 5, 1-7.
Ahmed, A., Singh, A., Young, S. J., Gupta, V., Singh, M., Arya, S., 2023. Synthesis techniques and advances in sensing applications of reduced graphene oxide (rGO) Composites: A review. Compos. Part A Appl. Sci. Manuf. 165, 107373.
Arbab Zavar, M. H., Heydari, S., Rounaghi, G. H., Eshghi, H., Azizi-Toupkanloo, H., 2012. Electrochemical behavior of para-nitroaniline at a new synthetic crown ether-silver nanoparticle modified carbon paste electrode. Anal. Methods 4, 953-958.
Bakhsh, E. M., Ali, F., Khan, S. B., Marwani, H. M., Danish, E. Y., Asiri, A. M., 2019. Copper nanoparticles embedded chitosan for efficient detection and reduction of nitroaniline. Int. J. Biol. Macromol. 131, 666-675.
Baran, T., Karaoğlu, K., Nasrollahzadeh, M., 2023. Nano-sized and microporous palladium catalyst supported on modified chitosan/cigarette butt composite for treatment of environmental contaminants. Environ. Res. 220.
Caliskan, S., Wang, A., Qin, F., House, S. D., Lee, J. K., 2022. Molybdenum Carbide-Reduced Graphene Oxide Composites as Electrocatalysts for Hydrogen Evolution. ACS Appl. Nano Mater. 5, 3790-3798.
Cao, X. N., Li, J. H., Xu, H. H., Zhan, J. R., Lin, L., Yamamoto, K., Jin, L. T., 2004. Simultaneous Determination of Aromatic Amines by Liquid Chromatography Coupled with Carbon Nanotubes/Poly (3-methylthiophene) Modified Dual-Electrode. Chromatographia 59, 167-172.
Chakraborty, G., Das, P., Mandal, S. K., 2018. Strategic Construction of Highly Stable Metal—Organic Frameworks Combining Both Semi-Rigid Tetrapodal and Rigid Ditopic Linkers: Selective and Ultrafast Sensing of 4-Nitroaniline in Water.
Chen, M., Zhang, C., Li, X., Zhang, Lei, Ma, Y., Zhang, Li, 2013. Journal of Materials Chemistry A 2869-2877.
Cheng, C., Li, S., Thomas, A., Kotov, N. A., Haag, R., 2017. Functional Graphene Nanomaterials Based Architectures: Biointeractions, Fabrications, and Emerging Biological Applications. Chem. Rev. 117, 1826-1914.
Coy, E., Yate, L., Valencia, D. P., Aperador, W., Siuzdak, K., Torruella, P., Azanza, E., Estrade, S., Iatsunskyi, I., Peiro, F., Zhang, X., Tejada, J., Ziolo, R. F., 2017. High Electrocatalytic Response of a Mechanically Enhanced NbC Nanocomposite Electrode Toward Hydrogen Evolution Reaction. ACS Appl. Mater. Interfaces 9, 30872-30879.
Das, P., Chakraborty, G., Mandal, S. K., 2020. Comprehensive Structural and Microscopic Characterization of an Azine-Triazine-Functionalized Highly Crystalline Covalent Organic Framework and Its Selective Detection of Dichloran and 4-Nitroaniline. ACS Appl. Mater. Interfaces.
de Barros, M. R., Winiarski, J. P., Elias, W. C., de Campos, C. E. M., Jost, C. L., 2021. Au-on-Pd bimetallic nanoparticles applied to the voltammetric determination and monitoring of 4-nitroaniline in environmental samples. J. Environ. Chem. Eng. 9.
Dong, L., Zhang, W., Fu, Y., Lu, J., Liu, X., Tian, N., Zhang, Y., 2021. Reduced Graphene Oxide Nanosheets Decorated with Copper and Silver Nanoparticles for Achieving Superior Strength and Ductility in Titanium Composites. ACS Appl. Mater. Interfaces.
Elgrishi, N., Rountree, K. J., McCarthy, B. D., Rountree, E. S., Eisenhart, T. T., Dempsey, J. L., 2018. A Practical Beginner's Guide to Cyclic Voltammetry. J. Chem. Educ. 95, 197-206.
Fazzo, L., Minichilli, F., Santoro, M., Ceccarini, A., Della Seta, M., Bianchi, F., Comba, P., Martuzzi, M., 2017. Hazardous waste and health impact: A systematic review of the scientific literature. Environ. Heal. A Glob. Access Sci. Source 16, 1-11.
Gao, Q., Zhang, W., Shi, Z., Yang, L., Tang, Y., 2019. Structural Design and Electronic Modulation of Transition-Metal-Carbide Electrocatalysts toward Efficient Hydrogen Evolution. Adv. Mater. 31, 1-35.
Gautam, S., Kamble, S. P., Sawant, S. B., Pangarkar, V. G., 2005. Photocatalytic degradation of 4-nitroaniline using solar and artificial UV radiation. Chem. Eng. J. 110, 129-137.
Gong, Y., Li, D., Fu, Q., Pan, C., 2015. Influence of graphene microstructures on electrochemical performance for supercapacitors. Prog. Nat. Sci. Mater. Int. 25, 379-385.
Grove, D. E., Gupta, U., Castleman, A. W., 2010. Effect of hydrocarbons on the morphology of synthesized niobium carbide nanoparticles. Langmuir 26, 16517-16521.
Guerrero-Contreras, J., Caballero-Briones, F., 2015. Graphene oxide powders with different oxidation degree, prepared by synthesis variations of the Hummers method. Mater. Chem. Phys. 153, 209-220.
Guex, L. G., Sacchi, B., Peuvot, K. F., Andersson, R. L., Pourrahimi, A. M., Strom, V., Farris, S., Olsson, R. T., 2017. Experimental review: Chemical reduction of graphene oxide (GO) to reduced graphene oxide (rGO) by aqueous chemistry. Nanoscale 9, 9562-9571.
Guo, X., Lv, J., Zhang, W., Wang, Q., He, P., Fang, Y., 2006. Separation and determination of nitroaniline isomers by capillary zone electrophoresis with amperometric detection. Talanta 69, 121-125.
Gupta, A., Mittal, M., Singh, M. K., Suib, S. L., Pandey, O. P., 2018. Low temperature synthesis of NbC/C nano-composites as visible light photoactive catalyst. Sci. Rep. 8, 1-17.
Halankar, K. K., Mandal, B. P., Nigam, S., Majumder, C., Srivastava, A. P., Agarwal, R., Tyagi, A. K., 2021. Experimental and Theoretical Study on rGO-Decorated Mo2C Composite as the Anode Material for Lithium Ion Batteries. Energy and Fuels 35, 12556-12568.
Hwa, K. Y., Santhan, A., Ganguly, A., Kanna Sharma, T. S., 2021. Synthesis of Nickel Vanadate Anchored on Reduced Graphene Oxide for Electrochemical Determination of Antioxidant Radical Cations of Diphenylamine H·+. ACS Appl. Electron. Mater. 3, 2247-2260.
Hwu, H. H., Chen, J. G., 2005. Surface chemistry of transition metal carbides. Chem. Rev. 105, 185-212.
Karunanayake, A. G., Bombuwala Dewage, N., Todd, O. A., Essandoh, M., Anderson, R., Mlsna, T., Mlsna, D., 2016. Salicylic Acid and 4-Nitroaniline Removal from Water Using Magnetic Biochar: An Environmental and Analytical Experiment for the Undergraduate Laboratory. J. Chem. Educ. 93, 1935-1938.
Kimmel, Y. C., Xu, X., Yu, W., Yang, X., Chen, J. G., 2014. Trends in electrochemical stability of transition metal carbides and their potential use as supports for low-cost electrocatalysts. ACS Catal. 4, 1558-1562.
Klug, J. A., Proslier, T., Elam, J. W., Cook, R. E., Hiller, J. M., Claus, H., Becker, N. G., Pellin, M. J., 2011. Atomic layer deposition of amorphous niobium carbide-based thin film superconductors. J. Phys. Chem. C 115, 25063-25071.
Kokulnathan, T., Ahmed, F., Chen, S., Chen, T., Hasan, P. M. Z., Bilgrami, A. L., Darwesh, R., 2021a. Rational Confinement of Yttrium Vanadate within Three-Dimensional Graphene Aerogel: Electrochemical Analysis of Monoamine Neurotransmitter (Dopamine).
Kokulnathan, T., Kumar, E. A., Wang, T. J., 2020. Design and in situ synthesis of titanium carbide/boron nitride nanocomposite: Investigation of electrocatalytic activity for the sulfadiazine sensor. ACS Sustain. Chem. Eng. 8, 12471-12481.
Kokulnathan, T., Wang, T. J., 2020. Vanadium Carbide-Entrapped Graphitic Carbon Nitride Nanocomposites: Synthesis and Electrochemical Platforms for Accurate Detection of Furazolidone. ACS Appl. Nano Mater. 3, 2554-2561.
Kokulnathan, T., Wang, T. J., Ahmed, F., 2021b. Construction of two-dimensional molybdenum carbide based electrocatalyst for real-time monitoring of parathion-ethyl. J. Environ. Chem. Eng. 9, 106537.
Kokulnathan, T., Wang, T. J., Ahmed, F., Alshahrani, T., 2023. Hydrothermal synthesis of ZnCr-LDH/Tungsten carbide composite: A disposable electrochemical strip for mesalazine analysis. Chem. Eng. J. 451, 138884.
Kumar, R., Joanni, E., Singh, R. K., Singh, D. P., Moshkalev, S. A., 2018. Recent advances in the synthesis and modification of carbon-based 2D materials for application in energy conversion and storage. Prog. Energy Combust. Sci. 67, 115-157.
Laghrib, F., Ajermoun, N., Bakasse, M., Lahrich, S., El Mhammedi, M. A., 2019a. Synthesis of silver nanoparticles assisted by chitosan and its application to catalyze the reduction of 4-nitroaniline. Int. J. Biol. Macromol. 135, 752-759.
Laghrib, F., Ajermoun, N., Hrioua, A., Lahrich, S., Farahi, A., El Haimouti, A., Bakasse, M., El Mhammedi, M. A., 2019b. Investigation of voltammetric behavior of 4-nitroaniline based on electrodeposition of silver particles onto graphite electrode. Ionics (Kiel). 25, 2813-2821.
Laghrib, F., Boumya, W., Lahrich, S., Farahi, A., El Haimouti, A., El Mhammedi, M. A., 2017. Electrochemical evaluation of catalytic effect of silver in reducing 4-nitroaniline: Analytical application. J. Electroanal. Chem. 807, 82-87.
Laghrib, F., Farahi, A., Bakasse, M., Lahrich, S., El Mhammedi, M. A., 2019c. Voltammetric determination of nitro compound 4-nitroaniline in aqueous medium at chitosan gelified modified carbon paste electrode (CS@CPE). Int. J. Biol. Macromol. 131, 1155-1161.
Lee, S. P., Ali, G. A. M., Hegazy, H. H., Lim, H. N., Chong, K. F., 2021. Optimizing Reduced Graphene Oxide Aerogel for a Supercapacitor.
Li, Q., Li, Q., Chen, D., Chen, D., Miao, J., Miao, J., Lin, S., Lin, S., Yu, Z., Yu, Z., Han, Y., Yang, Z., Zhi, X., Cui, D., Cui, D., An, Z., 2020. Ag-Modified 3D Reduced Graphene Oxide Aerogel-Based Sensor with an Embedded Microheater for a Fast Response and High-Sensitive Detection of NO2. ACS Appl. Mater. Interfaces 12, 25243-25252.
Li, Z., Chen, G., Deng, J., Li, D., Yan, T., An, Z., Shi, L., Zhang, D., 2019. Creating Sandwich-like Ti3C2/TiO2/rGO as Anode Materials with High Energy and Power Density for Li-Ion Hybrid Capacitors. ACS Sustain. Chem. Eng. 7, 15394-15403.
Liu, T. R., Chang, Y. C., Bayeh, A. W., Wang, K. C., Chen, H. Y., Wang, Y. M., Chiang, T. C., Tang, M. T., Tseng, S. C., Huang, H. C., Wang, C. H., 2020. Synergistic effects of niobium oxide-niobium carbide-reduced graphene oxide modified electrode for vanadium redox flow battery. J. Power Sources 473, 228590.
Ma, C. A., Chen, Z. Y., Lin, W. F., Zhao, F. M., Shi, M. Q., 2012. Template-free environmentally friendly synthesis and characterization of unsupported tungsten carbide with a controllable porous framework. Microporous Mesoporous Mater. 149, 76-85.
Ma, J., Du, Y., 2008. Synthesis of nanocrystalline hexagonal tungsten carbide via co-reduction of tungsten hexachloride and sodium carbonate with metallic magnesium. J. Alloys Compd. 448, 215-218.
Mahle, R., Mahapatra, P. L., Singh, A. K., Kumbhakar, P., Paliwal, M., Tiwary, C. S., Banerjee, R., 2022. Anaerobe Syntrophic Co-culture-Mediated Green Synthesis of Ultrathin Niobium Carbide (NbC) Sheets for Flexoelectricity Generation. ACS Sustain. Chem. Eng. 10, 13650-13660.
Manavalan, S., Veerakumar, P., Chen, S. M., Murugan, K., Lin, K. C., 2019. Binder-Free Modification of a Glassy Carbon Electrode by Using Porous Carbon for Voltammetric Determination of Nitro Isomers. ACS Omega 4, 8907-8918.
Manna, B., Raj, C. R., 2018. Nanostructured Sulfur-Doped Porous Reduced Graphene Oxide for the Ultrasensitive Electrochemical Detection and Efficient Removal of Hg (II). ACS Sustain. Chem. Eng. 6, 6175-6182.
Medeiros, F. F. P., Da Silva, A. G. P., De Souza, C. P., 2002. Synthesis of niobium carbide at low temperature and its use in hardmetal. Powder Technol. 126, 155-160.
Nataraj, N., Chen, S.-M., 2021. Interfacial Influence of Strontium Niobium Engulfed Reduced Graphene Oxide Composite for Sulfamethazine Detection: Employing an Electrochemical Route in Real Samples. J. Electrochem. Soc. 168, 057512.
Nataraj, N., Chen, T. W., Akilarasan, M., Chen, S. M., Lou, B. S., Al-onazi, W. A., Ajmal Ali, M., Elshikh, M. S., 2022. In-situ construction of ternary metal oxide heterostructures Mn@LaZrO: A novel multi-functional nanocatalyst for detecting environmental hazardous 4-nitroaniline. Chem. Eng. J. 446, 137025
Nataraj, N., Chen, T. W., Chen, S. M., Rwei, S. P., 2020. An efficient electrochemical sensor based on zirconium molybdate decorated reduced graphene oxide for the detection of hydroquinone. Int. J. Electrochem. Sci. 15, 8321-8335.
Neyens, E., Baeyens, J., 2003. A review of classic Fenton's peroxidation as an advanced oxidation technique. J. Hazard. Mater. 98, 33-50.
Niazi, A., Ghasemi, J., Yazdanipour, A., 2007. Simultaneous spectrophotometric determination of nitroaniline isomers after cloud point extraction by using least-squares support vector machines. Spectrochim. Acta-Part A Mol. Biomol. Spectrosc. 68, 523-530.
Nundy, S., Ghosh, A., Nath, R., Paul, A., Ali, A., Mallick, T. K., 2021. Reduced graphene oxide (rGO) aerogel: Efficient adsorbent for the elimination of antimony (III) and (V) from wastewater. J. Hazard. Mater. 420, 126554.
Palpandi, K., Raman, N., 2020. Electrochemical detection of 2-nitroaniline at a novel sphere-like Co2SnO4 modified glassy carbon electrode. New J. Chem. 44, 8454-8462.
Pfeifer, R., Tamiasso-Martinhon, P., Sousa, C., Moreira, J. C., Do Nascimento, M. A. C., Barek, J., 2016. Differential pulse voltammetric determination of 4-nitroaniline using a glassy carbon electrode: Comparative study between cathodic and anodic quantification. Monatshefte fur Chemie 147, 111-118.
Pg, E., Ojpcjvn, Q., Izcsje, D. S., Boe, Q., Tubcjmjuz, D., Uifsf, F., Bo, J. T., 1998. Vmusbijhi Sbuf Dbqbcjmjuz.
Qin, T., Wang, Z., Wang, Y., Besenbacher, F., Otyepka, M., Dong, M., 2021. Recent Progress in Emerging Two-Dimensional Transition Metal Carbides, Nano-Micro Letters. Springer Singapore.
Santhan, A., Hwa, K. Y., 2023. Construction of 2D niobium carbide-embedded silver/silver phosphate as sensitive disposable electrode material for epinephrine detection in biological real samples. Mater. Today Chem. 27, 101332.
Santhan, A., Hwa, K. Y., 2022a. Zinc Phosphate-Incorporated Niobium Carbide as an Effective Electrocatalyst for Ultrasensitive and Selective Monitoring of Monoamine Neurotransmitter. ACS Sustain. Chem. Eng.
Santhan, A., Hwa, K. Y., 2022b. Rational Design of Nanostructured Copper Phosphate Nanoflakes Supported Niobium Carbide for the Selective Electrochemical Detection of Melatonin. ACS Appl. Nano Mater. 5, 18256-18269.
Santhan, A., Hwa, K. Y., Ganguly, A., 2022. Self-assembled nanorods with reduced graphene oxide as efficient nano-catalyst for dual modality sensing of hazardous phenolic compound. Chemosphere 307, 135715.
Selvi, S. V., Nataraj, N., Chen, S. M., 2020. The electro-catalytic activity of nanosphere strontium doped zinc oxide with rGO layers screen-printed carbon electrode for the sensing of chloramphenicol. Microchem. J. 159, 105580.
Shafi, A., Bano, S., Sharma, L., Halder, A., Sabir, S., Khan, M. Z., 2022. Exploring multifunctional behaviour of g-C3N4 decorated BiVO4/Ag2CO3 hierarchical nanocomposite for simultaneous electrochemical detection of two nitroaromatic compounds and water splitting applications. Talanta 241, 123257.
Shi, L., Gu, Y., Chen, L., Yang, Z., Ma, J., Qian, Y., 2005. Synthesis and oxidation behavior of nanocrystalline niobium carbide. Solid State Ionics 176, 841-843.
Silambarasan, S., Vangnai, A. S., 2016. Biodegradation of 4-nitroaniline by plant-growth promoting Acinetobacter sp. AVLB2 and toxicological analysis of its biodegradation metabolites. J. Hazard. Mater. 302, 426-436.
Tong, C., Guo, Y., Liu, W., 2010. Simultaneous determination of five nitroaniline and dinitroaniline isomers in wastewaters by solid-phase extraction and high-performance liquid chromatography with ultraviolet detection. Chemosphere 81, 430-435.
Tong, Y., He, M., Zhou, Y., Nie, S., Zhong, X., Fan, L., Huang, T., Liao, Q., Wang, Y., 2018. Three-Dimensional Hierarchical Architecture of the TiO2/Ti3C2Tx/RGO Ternary Composite Aerogel for Enhanced Electromagnetic Wave Absorption. ACS Sustain. Chem. Eng. 6, 8212-8222.
Wang, S., Shao, L., Yu, L., Guan, J., Shi, X., Sun, Z., Cai, J., Huang, H., Trukhanov, A., 2021. Niobium Carbide as a Promising Pseudocapacitive Sodium-Ion Storage Anode. Energy Technol. 9, 1-8.
Weidman, M. C., Esposito, D. V., Hsu, Y. C., Chen, J. G., 2012. Comparison of electrochemical stability of transition metal carbides (WC, W 2C, Mo 2C) over a wide pH range. J. Power Sources 202, 11-17.
Yamuna, A., Chen, T. W., Chen, S. M., Ling Wu, W., 2021a. Simultaneous electrochemical determination of nitroaniline and flutamide based on iron vanadate and lanthanum vanadate nanocomposite modified electrode by voltammetric technique. J. Electroanal. Chem. 901, 115772.
Yamuna, A., Jiang, T. Y., Chen, S. M., 2021b. Preparation of K+ intercalated MnO2-rGO composite for the electrochemical detection of nitroaniline in industrial wastewater. J. Hazard. Mater. 411, 125054.
Yuan, S., Pang, S. Y., Hao, J., 2020. 2D transition metal dichalcogenides, carbides, nitrides, and their applications in supercapacitors and electrocatalytic hydrogen evolution reaction. Appl. Phys. Rev. 7, 15-18.
Zakaria, M. B., Zheng, D., Apfel, U. P., Nagata, T., Kenawy, E. R. S., Lin, J., 2020. Dual-Heteroatom-Doped Reduced Graphene Oxide Sheets Conjoined CoNi-Based Carbide and Sulfide Nanoparticles for Efficient Oxygen Evolution Reaction. ACS Appl. Mater. Interfaces 12, 40186-40193.
Zhang, W., Li, Z., Li, H., Li, W., Peng, S., Li, Y., 2022. Facile Synthesis of Amorphous NiO/Reduced Graphene Oxide as a Cocatalyst for Enhanced Dye-Sensitized Photocatalytic H2Evolution. Energy and Fuels 36, 15112-15119.
Zhao, C., Wang, Q., Zhang, H., Passerini, S., Qian, X., 2016. Two-Dimensional Titanium Carbide/RGO Composite for High-Performance Supercapacitors. ACS Appl. Mater. Interfaces 8, 15661-15667.

What is claimed is:

1. A composite material, called as NC/rGO-A, comprising niobium carbide (NC) and reduced graphene oxide aerogel (rGO-A) at a ratio of about 1:1.

2. The composite material NC/rGO-A of claim 1, wherein the niobium carbide (NC) has characteristic diffraction peaks at diffraction angle (20) of 35.1°, 40.6°, 58.5°, 69.9°, and 73.5°.

3. The composite material NC/rGO-A of claim 1, wherein the reduced graphene oxide aerogel (rGO-A) has characteristic diffraction peaks at diffraction angle (20) of 25.4° and 43.5°.

4. The composite material NC/rGO-A of claim 1, comprising
50%-60% of niobium,
20%-30% carbon, and
10%-20% of oxygen.

5. The composite material NC/rGO-A of claim 4, comprising
56.9% of niobium,
29.6% carbon, and
13.5% of oxygen.

6. A method for preparing the composite material NC/rGO-A of claim 1, comprising the steps of:
(i) dissolving niobium carbide and reduced graphene oxide aerogel in water as a mixture;
(ii) stirring to homogenize the mixture; and
(iii) sonicating the mixture thoroughly to produce the composite material NC/rGO-A.

7. A method for preparing an NC/rGO-A based modified electrode having great electrical conductivity, comprising the steps of:
(i) rinsing a bare screen-printed carbon electrode in distilled water and ethanol;
(ii) coating the composite material NC/rGO-A of claim 1 onto the rinsed bare screen-printed carbon electrode of step (i); and
(iii) drying the screen-printed carbon electrode with NC/rGO-A coating as obtained in step (ii).

8. The method of claim 7, wherein the screen-printed carbon electrode is dried in a hot air oven at about 50° C. in step (iii).

9. An NC/rGO-A based modified electrode, called as NC/rGO-A/SPCE, which comprises an electrode coated with the composite material NC/rGO-A of claim 1.

10. The NC/rGO-A/SPCE of claim 9, which is prepared by a method comprising the steps of
(i) rinsing a bare screen-printed carbon electrode in distilled water and ethanol;
(ii) coating the composite material NC/rGO-A onto the rinsed bare screen-printed carbon electrode of step (i); and
(iii) drying the screen-printed carbon electrode with NC/rGO-A coating as obtained in step (ii).

11. The NC/rGO-A/SPCE of claim 9, which is used for detection of organic pollutants in a contaminated water sample.

12. The NC/rGO-A/SPCE of claim 9, which has an excellent selectivity exposing to interfering compounds.

13. The NC/rGO-A/SPCE of claim 12, wherein the interfering compounds include 4-nitrophenol, aminophenol, nitrobenzene, acetaminophen, carbendazim, chlorine ions, sodium ions, glucose, mercury, and hydroquinone.

14. The NC/rGO-A/SPCE of claim 11, wherein the organic pollutants are nitroanilines (NTA).

15. The NC/rGO-A/SPCE of claim 14, wherein the nitroanilines (NTA) include 2-Nitroaniline (2-NTA), 3-Nitroaniline (3-NTA), and 4-Nitroaniline (4-NTA).

16. The NC/rGO-A/SPCE of claim 11, wherein the organic pollutant is 4-NTA.

17. The NC/rGO-A/SPCE of claim 11, wherein the contaminated water sample is collected from industrial waste water, river water, or lake water.
